(19) United States
(12) Patent Application Publication
(10) Pub. No.: US 2006/0111309 A1
(43) Pub. Date: May 25, 2006

(54) Chalcomycin derivatives

(75) Inventors: Werner Simon, Huffelsheim (DE); Rajendra Prasad Maskey, Heidelberg (DE); Hartmut Laatsch, Gottingen (DE); Elisabeth Helmke, Bremerhaven (DE); Friedrich Hansske, Hirschberg (DE)

Correspondence Address: CONNOLLY BOVE LODGE & HUTZ, LLP, P O BOX 2207, WILMINGTON, DE, 19899, US

(73) Assignee: Biofrontera Discovery GmbH, Heidelberg (DE)

(21) Appl. No.: 11/109,399

(22) Filed: Apr. 15, 2005

Related U.S. Application Data
(63) Continuation of application No. PCT/EP03/11549, filed on Oct. 17, 2003.

(30) Foreign Application Priority Data
Oct. 17, 2002 (DE) 102 48 453.8

(51) Int. Cl. A61K 31/7048; C07H 17/08

(52) U.S. Cl. 514/28; 536/7.1

(57) ABSTRACT

The invention relates to novel chalcomycin derivatives, medicaments containing said derivatives or the salts thereof, and the use of chalcomycin derivatives for treating diseases, especially infections. The inventive chalcomycin derivatives are acylated on the sugar esters and derivatives are also acylated on the 9-carbonyl function.

The invention relates to novel chalcomycin derivatives, to drugs containing said derivatives or the salts thereof, and to the use of the chalcomycin derivatives for treating diseases, particularly infections.

Chalcomycin is a macrolid antibiotic and can be isolated from streptomycetes, particularly from Streptomyces bikiniensis, and demonstrates antibiotic activity.

Chalcomycin and several chalcomycin derivatives such as 4-desoxychalcomycin and 10,11-desoxychalcomycin are known.

Surprisingly it was found that chalcomycin derivatives with acylated sugar residues represent potent drugs. This is also true for derivatives which are additionally modified at the 9-carbonyl function.

The invention relates to novel chalcomycin derivatives with the general Formula Ia to Ig: 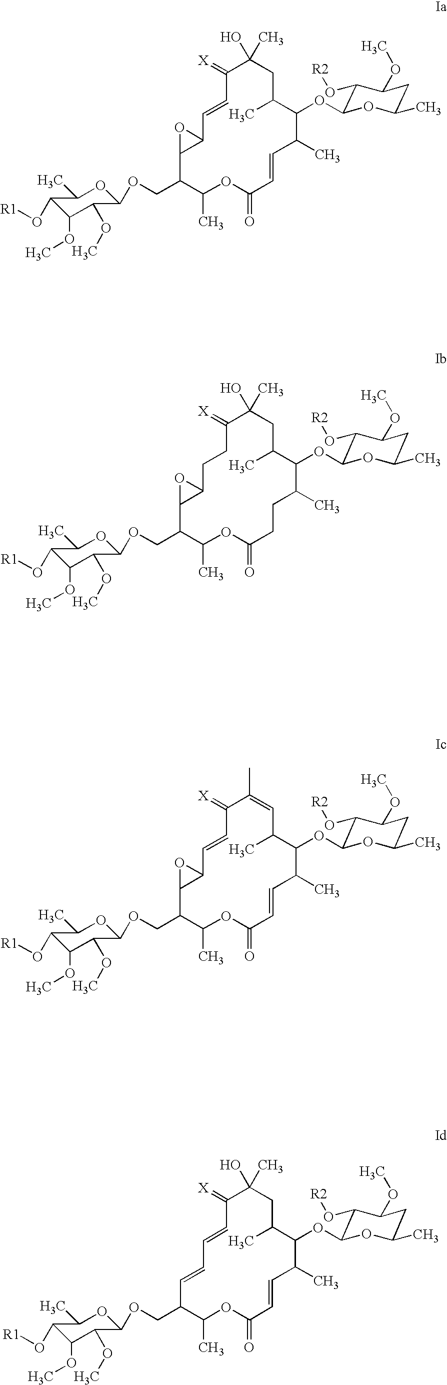 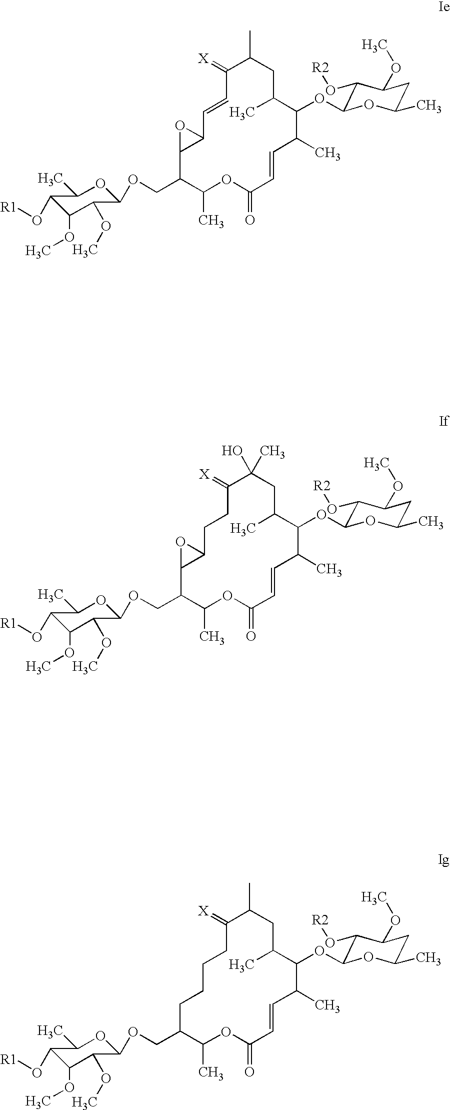
wherein in each,
R1 means H, CO—C₁-C₆ alkyl,
R2 means H, CO—C₁-C₆ alkyl,
X means O, NOH, NOR3, with
R3 being C₁-C₆ alkyl, C₂-C₆ alkenyl, aryl, C₁-C₄ alkyl aryl, heteroaryl, C₁-C₄ alkyl heteroaryl, cycloalkyl, C₁-C₄ alkylcycloalkyl, heterocycloalkyl, C₁-C₄ alkyl heterocycloalkyl,
   wherein R1 und R2 in formula Ia, Ie, If and Ig may not concomitantly be H, the stereoisomers, tautomeres thereof and their physiologically tolerable salts.

Preferred stereoisomers are those that correspond to the special structure of the natural chalcomycin and its derivatives.

Preferred are compounds of formula IIa to IIg 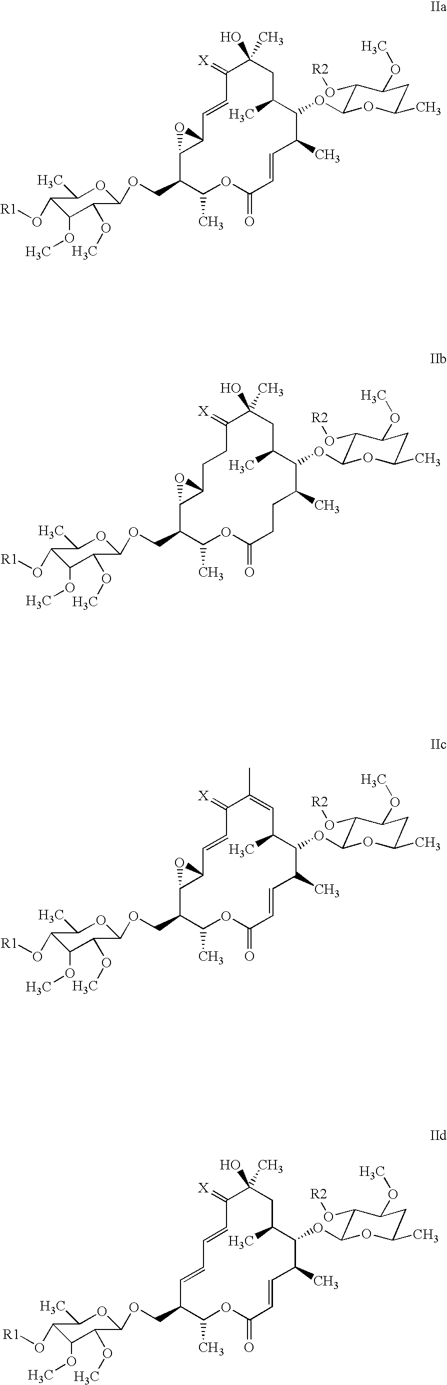 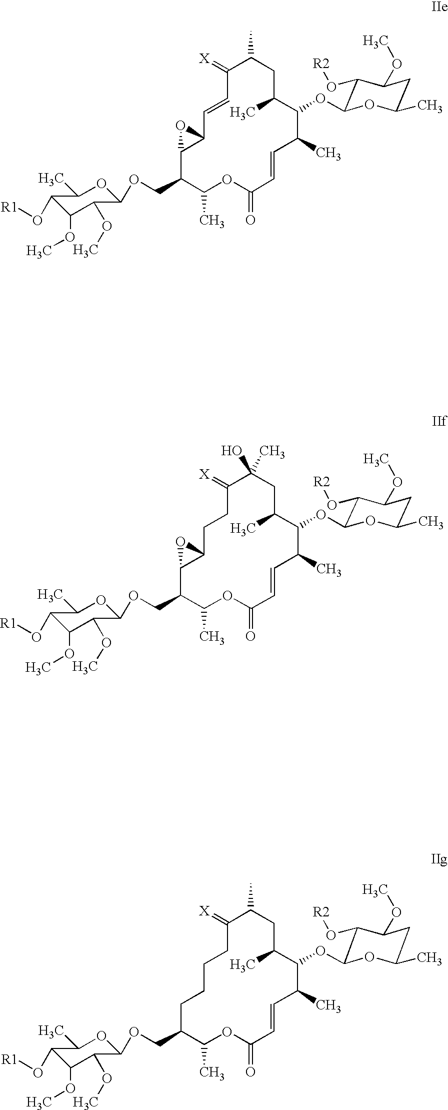
wherein the residues R1, R2 and X have the above described meanings of formula Ia to Ig.

Preferred are compounds of formula Ia to Ig and IIa to IIg, wherein residue R1 adopts the meaning CO—C₁-C₆ alkyl.

Preferred are further compounds of formula Ia to Ig and IIa to IIg, wherein residue R2 adopts the meaning CO—C₁-C₆ alkyl.

Particularly preferred are compounds of formula Ia to Ig and IIa to IIg, wherein residue R1 adopts the meaning COCH₃ or COCH₂CH₃, particularly COCH₂CH₃.

Equally particularly preferred are compounds of formula Ia to Ig and IIa to IIg, wherein residue R2 adopts the of meaning COCH₃ or COCH₂CH₃, particularly COCH₂CH₃.

Particularly preferred are compounds of formula Ia to Ig and IIa to IIg, wherein residue R1 and R2 adopt the same meaning.

Preferred are compounds of formula Ia to Ig and IIa to IIg, wherein residue X adopts the meaning O.

The invention furthermore relates to drugs containing the above compounds of formula I or II together with the usual carriers and adjuvants.

Also preferred are the above mentioned drugs in combination with other agents for treatment of infections.

These compounds according to the invention are used for preparation of drugs for the treatment or prevention of infections. Especially for the treatment of infections caused by gram-positive bacteria such as staphylococci, e.g. Staphylococcus aureus. The chalcomycin derivatives are also active against Bacillus subtilis.

In the description and the claims, the substituents are described by the following definitions:

The term “alkyl” by itself or as part of another substituent means a linear or branched alkyl chain radical of the respectively indicated length. Thus, C₁₋₄ alkyl may be methyl, ethyl, 1-propyl, 2-propyl, 2-methyl-2-propyl, 2-methyl-1-propyl, 1-butyl, 2-butyl, C₁₋₆ alkyl, e.g. C₁₋₄ alkyl, pentyl, 1-pentyl, 2-pentyl, 3-pentyl, 1-hexyl, 2-hexyl, 3-hexyl, 4-methyl-1-pentyl, or 3,3-dimethylbutyl.

The term “alkenyl” by itself or as part of another substituent means a linear or branched alkyl chain radical with one or more C═C double bonds of the respectively indicated length, several double bonds being preferably conjugated. Thus, C₂₋₆ alkenyl may for example be ethenyl, 1-propenyl, 2-propenyl, 2-methyl-2-propenyl, 2-methyl-1-propenyl, 1-butenyl, 2-butenyl, 1,3-butdienyl, 2,4-butdienyl, 1-pentenyl, 2-pentenyl, 3-pentenyl, 1,3-pentdienyl, 2,4-pentdienyl, 1,4-pentdienyl, 1-hexenyl, 2-hexenyl, 1,3-hediexyl, 4-methyl-1-pentenyl, or 3,3-dimethylbutenyl.

The term “halogen” stands for fluorine, chlorine, bromine, iodine, preferably bromine and chlorine.

The term “cycloalkyl” by itself or as part of another substituent comprises unsaturated (mono or poly, preferably mono) or saturated, cyclic carbohydrate groups with 3 to 10 C atoms, preferably 3 to 8 C atoms, such as e.g. cyclopropyl, cyclobutyl, cyclopentyl, cyclohexyl, cyclohex-2-enyl, cyclohex-3-enyl, cyclohex-2,4-dienyl, 4-methylcyclohexyl, 3-methylcyclohexyl, cycloheptyl or cyclooctyl. Saturated cycloalkyls are preferred. The cycloalkyls may be substituted with up to 3 substituents, preferably with up to 1 substituent, wherein the substituents independently of each other can have the meaning C₁-C₆ alkyl, OH, NO₂, CN, CF₃, OR11, SH, SR11, C₁-C₆ alkylhydroxy, C₁-C₆ alkyl-OR11, COOH, COOR11, NH₂, NHR11, NR11R12, halogen, aryl, C₁-C₄ alkylaryl, heteroaryl, C₁-C₄ heteroalkylaryl, wherein the residues R11 und R12 independently of each other can mean C₁-C₁₀ alkyl, cycloalkyl, C₁-C₄ alkylcycloalkyl.

The term “heterocycloalkyl” by itself or as part of another substituent includes cycloalkyl groups, wherein up to two CH₂ groups may be substituted by oxygen, sulfur or nitrogen atoms, and one or two other CH₂ groups may be substituted by one or two carbonyl function(s), carbothionyl function(s), or a carbonyl function and a carbothionyl function, for example pyrrolidine, piperidine, morpholine or

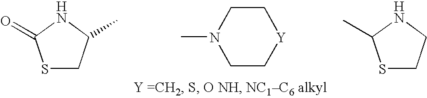

The heterocycloalkyls may be substituted as the cycloalkyls.

The term “aryl” by itself or as part of another substituent includes aromatic ring systems with up to 3 rings, in which at least 1 ring system is aromatic, and those with up to 3 substituents, preferably up to 1 substituent, wherein the substituents independently of each other can have the meaning C₁-C₆ alkyl, OH, NO₂, CN, CF₃, OR11, SH, SR11, C₁-C₆ alkylhydroxy, C₁-C₆ alkyl-OR11, COOH, COOR11, NHx₂, NHR11, NR11R12, halogen, wherein the residues R11 und R12 independently of each other can mean C₁-C₁₀ alkyl, cycloalkyl, C₁-C₄ alkylcycloalkyl.

Apart from phenyl and 1-naphthyl and 2-naphthyl, preferred aryls are:

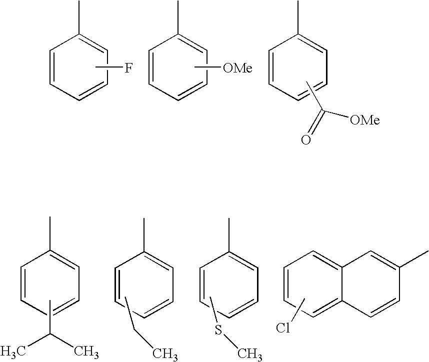

The term “heteroaryl” by itself or as part of another substituent includes aromatic ring systems with up to 3 rings and with up to 3 identical or different heteroatoms N, S, O, in which at least 1 ring system is aromatic, and those with up to 3 substituents, preferably up to 1 substituent, wherein the substituents independently of each other can have the meaning C₁-C₆ alkyl, OH, NO₂, CN, CF₃, OR11, SH, SR11, C₁-C₆ alkylhydroxy, C₁-C₆ alkyl-OR11, COOH, COOR11, NH₂, NHCOR11, NHR11, NR11R12, halogen, or phenyl, wherein the residues R11 und R12 independently can have the above indicated meanings.

Preferred heteroaryls are:

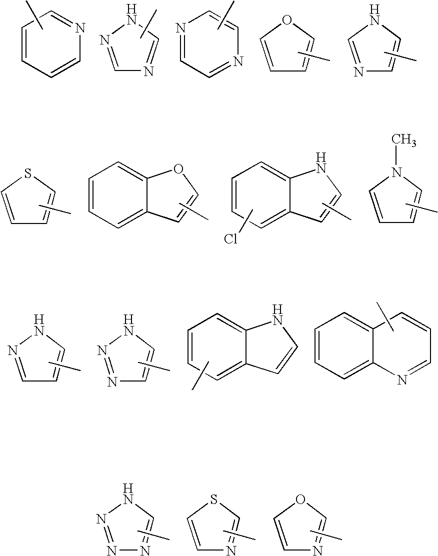

The term “ring system” generally refers to rings with 3, 4, 5, 6, 7, 8, 9, or 10 members. Preferred are rings with 5 and 6 members. Furthermore, ring systems with one or 2 annelated rings are preferred.

The compounds of formula I or II may be present as such, or, if they contain acidic or basic groups, in the form of their salts with physiologically tolerable bases or acids. Examples for such acids are: hydrochloric acid, citric acid, trifluoracetic acid, tartaric acid, lactic acid, phosphoric acid, methane sulfonic acid, acetic acid, formic acid, maleic acid, fumaric acid, succinic acid, hydroxysuccinic acid, sulfuric acid, glutaric acid, aspartic acid, pyruvic acid, benzoic acid, glucuronic acid, oxalic acid, ascorbic acid, and acetylglycine. Examples for bases are alkali ions, preferably Na, K, alkaline earth ions, preferably C, Mg, ammonium ions.

The compounds according to the invention may be administered orally in the usual way. The application may also be i.v., i.m., with vapors, or sprays through the nasopharynx.

The dosage depends on age, condition and weight of the patient as well as on the type of application. Usually, the daily dose of the active ingredient per person is between 0.1 μg/kg and 1 g/kg orally. This dosage may be given as 2 to 4 split dosages, or once per day as a slow release form.

The novel compounds may be used in the usual solid or liquid pharmaceutical application forms, e.g. as tablets, film tablets, capsules, powder, granules, coated tablets, solutions, or sprays. These are produced in the usual way. The agents can be processed with the usual pharmaceutical adjuvants such as tablet binders, fillers, preservatives, disintegrants, flow regulators, plasticizers, wetting agents, dispersants, emulsifiers, solvents, retardation agents, antioxidants, and/or propellants (compare H. Sucker et al.: Pharmazeutische Technologie, Thieme-Verlag, Stuttgart, 1978). Usually, the so obtained application forms contain the active ingredient in amounts of 0.1 to 99 percent per weight.

Experimental Part

The compounds of formula I and II may be prepared fully synthetically according to known methods. A more simple way is the semisynthetic preparation from accessible starting substances such as chalcomycin or known chalcomycin derivatives according to methods that are know per se, e.g. by acylation. For propionic acid esters this may occur, for example, with propionylchloride, propionic acid or propionic acid anhydride. Oxymation and desoxygenation may be performed according to standard methods as well.

For example, the substances according to the invention may be prepared by following synthesis:

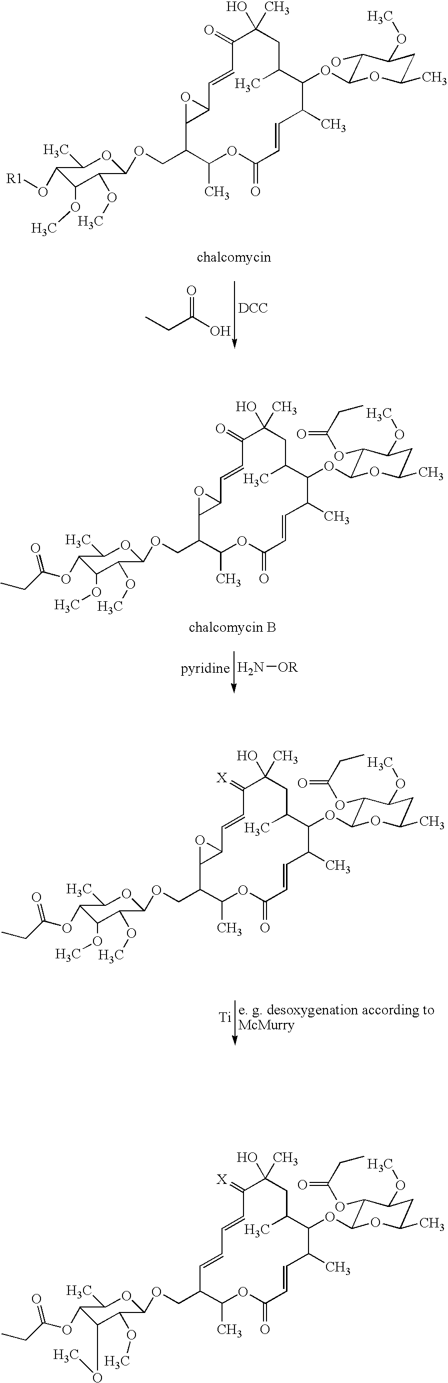

Chalcomycin (1a) and chalcomycin B (1b) (compound of formula Ia according to the invention, with R being ethyl) may be isolated or prepared according to the described methods. 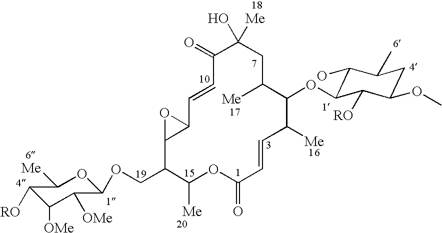
1a: R═H
1b: R=EtCO

The physical data of compound 1a and 1b are as follows.

TABLE 1
Physicochemical characteristics of the chalcomycins (1a and 1b)

| | Chalcomycin (1a) | Chalcomycin (1b) |
|---|---|---|
| Status | Colorless solid | Colorless solid |
| R_{f} | 0.55^{a} | 0.78^{a}, 0.60^{b} |
| Empirical formula | C₃₅H₅₆O₁₄ | C₄₁H₆₄O₁₆ |
| Melting point | 120-121° C. | 98° C. |
| (+) ESI MS | 723 ([M + Na]⁺), | 835 ([M + Na]⁺), |
| | 1423 ([2 M + Na]⁺) | 1647 ([2 M + Na]⁺) |
| ESI HRMS | | calc. 812.4194 |
| | | exp. 812.4191 |
| IR (KBr) | 3490, 2930, 1718, 1630, | 3445, 2935, 1740, 1730, |
| v cm⁻¹ | 1498, 1350, 1236, 1170, | 1634, 1459, 1355, 1178, |
| | 1083, 982, 890, 726 | 1080, 980, 850, 804 |

^{a}CHCl₃/10% MeOH,
^{b}CHCl₃/5% MeOH

TABLE 2
¹³C NMR data of chalcomycin (1a) and chalcomycin
B (1b) in deuteriochloroform (125 MHz)

| | Chemical | | | Chemical | | | Chemical | |
|---|---|---|---|---|---|---|---|---|
| | displacement | | | displacement | | | displacement | |
| C | (δ) | | C | (δ) | | C | (δ) | |
| atom | 1a | 1b | atom | 1a | 1b | atom | 1a | 1b |
| 1 | 165.3 | 165.3 | 15 | 68.9 | 68.8 | 8′ | — | 27.7 |
| 2 | 120.7 | 120.9 | 16 | 18.6 | 18.2 | 9′ | — | 9.1 |
| 3 | 151.8 | 151.3 | 17 | 19.2 | 19.1 | 1″ | 100.9 | 100.8 |
| 4 | 41.7 | 41.3 | 18 | 27.8 | 27.8 | 2″ | 81.9 | 80.1 |
| 5 | 87.8 | 88.5 | 19 | 66.9 | 67.1 | 2″- | 58.7 | 59.4 |
| | | | | | | OMe | | |
| 6 | 34.0 | 34.0 | 20 | 18.3 | 18.3 | 3″ | 79.6 | 77.6 |
| 7 | 37.0 | 37.1 | 1′ | 103.2 | 101.6 | 3″- | 61.7 | 61.6 |
| | | | | | | OMe | | |
| 8 | 78.4 | 78.4 | 2′ | 75.0 | 74.2 | 4″ | 72.9 | 74.6 |
| 9 | 200.2 | 200.0 | 3′ | 80.5 | 78.8 | 5″ | 70.7 | 67.4 |
| 10 | 124.8 | 125.0 | 3′- | 56.7 | 56.5 | 6″ | 17.8 | 17.4 |
| | | | OMe | | | | | |
| 11 | 146.5 | 146.3 | 4′ | 36.8 | 37.1 | 7″ | — | 173.6 |
| 12 | 59.7 | 58.7 | 5′ | 67.7 | 67.7 | 8″ | — | 27.6 |
| 13 | 59.0 | 59.0 | 6′ | 20.9 | 20.8 | 9″ | — | 9.2 |
| 14 | 49.5 | 49.4 | 7′ | — | 173.2 | | | |

TABLE 3
Antimicrobial activity of 1a, 1b and erythromycin (E)
in the agar diffusion test (diameter of the inhibition
zone in mm at 10 μg/9 mm Ø plate).

| | SA^{a} | EC^{b} | BS^{c} | CA^{d} | MM^{e} |
|---|---|---|---|---|---|
| 1a | 30 | 32 | 25 | 0 | 0 |
| 1b | 23 | 28 | 21 | 0 | 0 |
| E | 24 | 30 | 22 | — | — |

^{a}Stapylococcus aureus,
^{b}Escherichia coli,
^{b}Bacillus subtillis,
^{d}Candida albicans,
^{e}Mucor miehei

TABLE 4
Antimicrobial activity of 1a, 1b after conducting
the dilution method; MIC (μg/mL)

| | SA | EC | BS | CV^{a} | CS^{b} | SS^{c} |
|---|---|---|---|---|---|---|
| 1a | 0.39 | >50 | 6.25 | 50 | 50 | 50 |
| 1b | 0.39 | >50 | 6.25 | 50 | 50 | 50 |

^{a}Chlorella vulgaris,
^{b}Chlorella sorokiniana,
^{c}Scenedesmus subspicatus

1. A compound according to one of the general formula Ia to Ig: 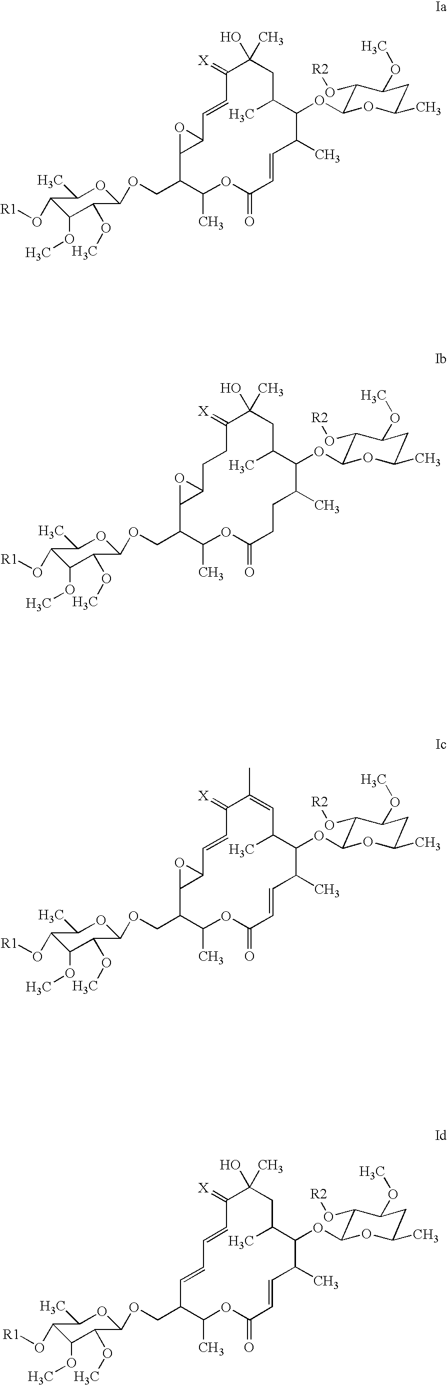 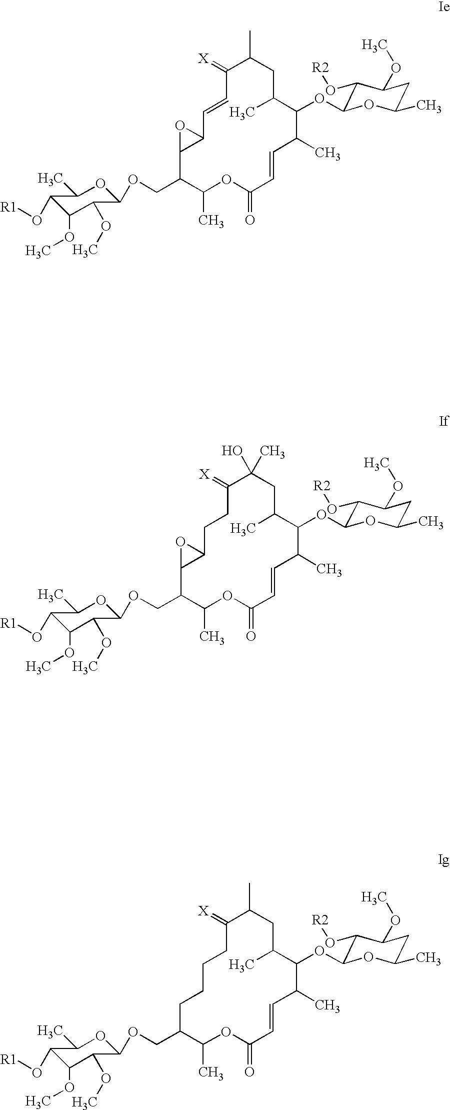
wherein in each
R1 is H, CO—C₁-C₆ alkyl,
R2 is H, CO—C₁-C₆ alkyl,
X is O, NOH, NOR3, wherein
R3 is C₁-C₆ alkyl, C₂-C₆ alkenyl, aryl, C₁-C₄ alkyl aryl, heteroaryl, C₁-C₄ alkyl heteroaryl, cycloalkyl, C₁-C₄ alkylcycloalkyl, heterocycloalkyl, C₁-C₄ alkyl heterocycloalkyl,
wherein R11 and R2 in formula Ia, Ie, If and Ig may not concomitantly be H, the stereoisomers, tautomeres thereof and their physiologically tolerable salts.

2. The compound of claim 1, wherein formula Ia to Ig adopt the stereochemistry of formula IIa to IIg

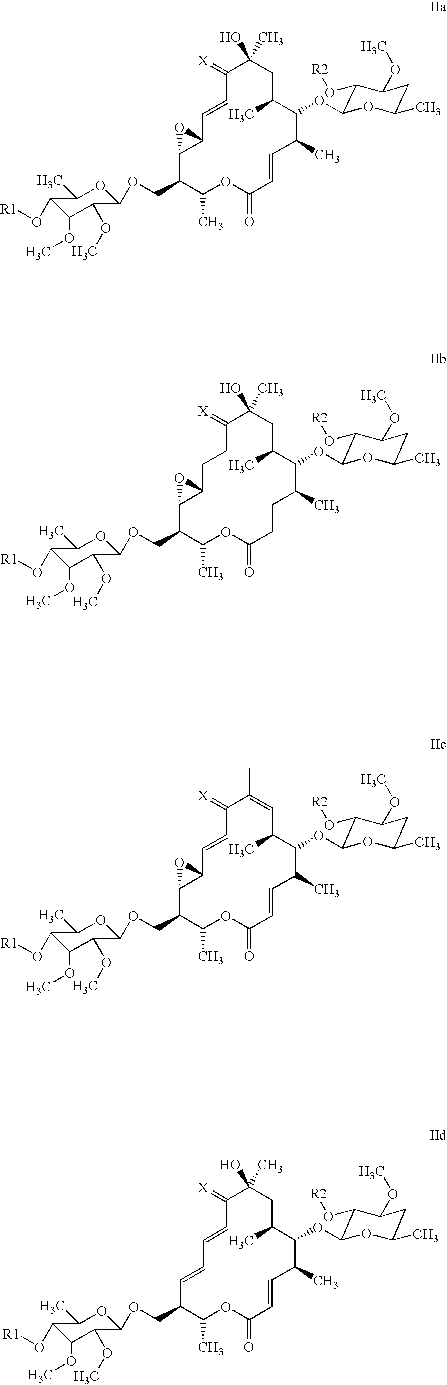

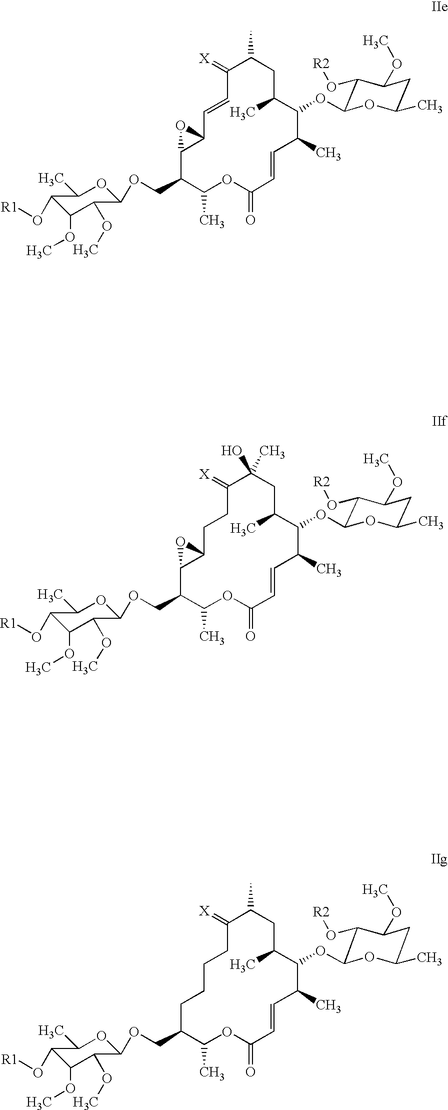

3. The compound of claim 1, wherein residue R1 is CO—C₁-C₆ alkyl.

4. The compound of claim 1, wherein residue R2 is CO—C₁-C₆ alkyl.

5. The compound of claim 1, wherein residue R1 is COCH₃ or COCH₂CH₃.

6. The compound of claim 1, wherein residue R2 is COCH₃ or COCH₂CH₃.

7. The compound of claim 1, wherein residues R1 and R2 are the same.

8. The compound of claim 1, wherein X is O.

9-11. (canceled)

12. The compound of claim 2, wherein residue R1 is CO—C₁-C₆ alkyl.

13. The compound of claim 2, wherein residue R2 is CO—C₁-C₆ alkyl.

14. The compound of claim 3, wherein R2 is CO—C I—C₆ alkyl.

15. The compound of claim 12, wherein R2 is CO—C₁-C₆ alkyl.

16. The compound of claim 2, wherein R1 is COCH₃ or COCH₂CH₃.

17. The compound of claim 2, wherein R2 is COCH₃ or COCH₂CH₃.

18. A pharmaceutical composition comprising a compound of claim 1 and a carrier or adjuvant.

19. The pharmaceutical composition of claim 18 further comprising another agent for the treatment of infections.

20. A method of treating infections comprising administering to a patient in need of such treatment an effective amount of a compound of claim 1.
